(19) United States
(12) Patent Application Publication
(10) Pub. No.: US 2025/0052754 A1
(43) Pub. Date: Feb. 13, 2025

(54) COMPOSITIONS AND METHODS FOR DIAGNOSIS, TREATMENT OF AND PREVENTION OF KIDNEY DISEASE

(71) Applicant: XORTX THERAPEUTICS, INC., Calgary (CA)

(72) Inventor: Allen DAVIDOFF, Calgary (CA)

(21) Appl. No.: 18/722,330

(22) PCT Filed: Dec. 20, 2022
(86) PCT No.: PCT/IB2022/000799

§ 371 Date: Jun. 20, 2024

Related U.S. Application Data
(60) Provisional application No. 63/422,551, filed on Nov. 4, 2022.
(60) Provisional application No. 63/291,851, filed on Dec. 20, 2021.

(51) Int. Cl. G01N 33/573; A61K 31/155; A61K 31/519; A61K 31/5517; A61K 45/06; A61P 13/12; C12Q 1/26

(52) U.S. Cl. CPC G01N 33/573; A61K 31/155; A61K 31/519; A61K 31/5517; A61K 45/06; A61P 13/12; C12Q 1/26; G01N 2333/9029; G01N 2800/347; G01N 2800/52

(57) ABSTRACT

Disclosed herein are methods of detecting xanthine oxidase expression in polycystic kidney disease, or methods of delaying the onset or treating polycystic kidney disease, or both. The inventors have made the remarkable discovery that aberrant purine metabolism is not a corollary to disease but rather tissue expression of xanthine oxidase/xanthine dehydrogenase (XO/XDH) expression is increased and possibly uric acid and oxygen radical production acting alone or in combination acts as a primary mediator of progression of structural and functional dysfunction. Specifically exemplified are methods that involve, measuring and evaluating tissue XO/XDH expression in polycystic kidney disease or any cystic tissue. Moreover, specifically exemplified are methods that involve administering to a patient susceptible to development of polycystic kidney disease a composition of agents, including uric acid lowering agents in a regimen that decreases expression of XO/XDH, lowering intracellular uric acid or free oxygen radical expression in a tissue.

### BACKGROUND

Polycystic Kidney disease is characterized by an array of anatomic and physiologic abnormalities, for example hypertension, endothelial dysfunction, cardiovascular disease, liver disease, aberrations involving large, medium and small caliber blood vessels and other health problems encountered by patients with polycystic kidney disease. Polycystic kidney disease has two primary causes that are genetic—autosomal dominant polycystic kidney disease (ADPKD) and autosomal recessive polycystic kidney disease (ARPKD) classified into two categories. Other causes of polycystic kidney disease or cystic disease syndrome can be found associated with cystic fibrosis, renal dysplasia, tuberous sclerosis, Von Hippel-Lindau disease, multicystic dysplastic kidney (MCDK), medullary sponge kidney, and acquired renal cystic disease. While the origin of polycystic kidney disease is often genetic and the primary cause of the disease, approaches to diagnose and treat existing disease can be categorized as primary prevention, secondary prevention and tertiary prevention. Often progression of disease is variable, and some, but not all identifiable conditions cause or allow polycystic kidney disease to develop, for example, 1) pancreatic disease, 2) hormonal abnormalities, 3) drug or chemical induced, 4) tubule abnormalities, 5) genetic syndromes and 6) others.

The two primary causes of polycystic kidney disease are ADPKD and ARPKD, both are genetic diseases contributing approximately 90% and 5% of all primary polycystic kidney disease, respectively. Frequently associated with polycystic kidney disease are symptoms that may or may not include hypertension, cardiovascular disease, endothelial dysfunction, hyperuricemia, gout, kidney stones, hematuria, abdominal pain, high frequency of urinary tract infection, headache, abnormal cardiac valve structure, purpura, fatigue and nausea and vomiting.

Many forms of kidney disease progress with an accelerating loss of filtering capacity.

### SUMMARY

The embodiments described herein are based on the remarkable discovery that increased serum concentrations of uric acid in polycystic kidney disease are a primary mediator of increased kidney size and decreased kidney function. In addition, the inventors have made the remarkable discovery that increased expression and increased activity of xanthine oxidase/xanthine dehydrogenase can be found in the polycystic kidney and can act as a mediator kidney injury. Also described is the discovery that the polycystic kidney uniquely expresses a modulator of injury, different from the effects of circulating serum uric acid and that targeting tissue expression of xanthine oxidase or modulators of Xanthine Oxidase expression represents a novel opportunity to target this newly discovered mechanism of injury. Aspects of the invention provide a new approach to both diagnosing and combating the progressive decline of structural integrity and function of the polycystic kidney.

In a specific embodiment, the subject of the invention pertains to methods of a detecting increased tissue expression of xanthine oxidase (XO), xanthine dehydrogenase (XDH) expression of both in an individual susceptible to polycystic disease.

In a specific embodiment, the subject of the invention pertains to methods of detecting increased tissue expression of a biological signaling molecule such as a modulator of cytokine expression, inflammatory system, and/or proteome expression that is modulated by increase XO/XDH expression in an individual susceptible to progressing or accelerated progression of polycystic kidney disease.

In a specific embodiment, provided are methods of detecting, stratifying, identifying and treating individuals whose tissue XO/XDH places them at increased risk of polycystic kidney disease. As a method of the treatment, serum or urine samples may be obtained and tested so that serum uric acid, or extracellular vesicles or cellular membrane components may be tested to determine XO/XDH over expression and may be monitored in conjunction with the administration of a uric acid lowering agent.

In another embodiment, provided are compositions and formulations of uric acid lowering agents that would provide improved absorption or bioavailability of a uric acid lowering agent and its dose, route or regimen of delivery. As a method of the treatment, serum or urine samples may be obtained and tested so that serum uric acid, or extracellular vesicles or cellular membrane components may be tested to determine XO/XDH over expression and may be monitored in conjunction with the administration of a uric acid lowering agent and/or an agent that decreases expression of XO/XDH.

In another embodiment, provided are methods of decreasing expression of xanthine oxidase or xanthine dehydrogenase in a tissue using an inhibitory RNA (iRNA or siRNA), more specifically in a kidney. As a method of the treatment, serum or urine samples may be obtained and tested so that serum uric acid, or extracellular vesicles or cellular membrane components may be tested to determine XO/XDH over expression and may be monitored in conjunction with the administration of a uric acid lowering agent.

In another embodiment, methods are provided involving delivery of an iRNA or siRNA to a tissue where over expression of XO or XDH occurs to target the administration of a therapy to a desired tissue or tissues. As a method of the treatment, serum or urine samples may be obtained and tested so that serum uric acid, or extracellular vesicles or cellular membrane components may be tested to determine XO/XDH over expression and may be monitored in conjunction with the administration of a uric acid lowering agent.

In another embodiment, the provided are methods of reducing the risk of developing, delaying the onset of and/or treating progressive kidney disease, and more specifically polycystic kidney disease.

In another embodiment, provided are methods of reducing the risk of developing, delaying the onset of and/or treating progressive liver disease, and more specifically polycystic liver disease.

In another embodiment, the subject of the invention provides a method of specifically treating a polycystic kidney to reduce injury associated with tissue specific expression of xanthine oxidase or XO/XDH.

Another embodiment relates to a method of specifically treating a cystic disease in a tissue to reduce injury associated with tissue specific expression of XO, XDH or XO/XDH expression, in combination with xanthine oxidase inhibitor, and an agent capable of decreasing or modulating XO/XDH expression in a tissue.

Also provided is method of specifically treating a cystic disease in a tissue increase expression of an upstream modulator, e.g., Sirtuin expression, and more specifically Sirtuin-1 expression as a means of modulating tissue specific expression of XO/XDH.

Yet another embodiment pertains to a composition comprising gliflozin and an xanthine oxidase inhibitor. It is believed that gliflozin increases the aqueous solubility of a xanthine oxidase inhibitor, the oral bio-availablity of that xanthine oxidase inhibitor. Also, as is described herein, gliflozin also has further functional benefits that make its use in combination with other UALAs particularly beneficial in the context of cystic kidney disease.

### BRIEF DESCRIPTION OF THE DRAWINGS

FIG. 1: provides a schematic depicting purine metabolism and purine based xanthine oxidase inhibitor metabolism.

FIG. 2: provides a diagram depicting modulators of Xanthine Oxidase/Xanthine Dehydrogenase.

FIG. 3: provides a graph showing that animal models of polycystic kidney disease exhibit high serum concentrations of allantoin.

FIG. 4: provides data showing that kidney tissue expression of XO is increased in a rodent model of polycystic kidney disease. FIG. 4A provides a western blot showing increased expression of xanthine oxidase. FIG. 4B Provides Data from a rat model of polycystic kidney disease Han/SPDR (CY/+) or wild type rat kidney. Light microscopy of kidney tissue sections, stained to show the presence of the xanthine oxidase enzyme, and reads as relative densitometric Units (RD). Han/SPDR show an increased proportion of cytoplasm stained compared to wildtype (p<0.05) 4C Provides evidence of a similar observation in the mouse polycystic kidney disease model (Pkd1 RC/RC mouse) (P<0.05).

FIG. 5: is a graph showing that XO activity is increased in a rodent model of polycystic kidney disease kidney tissue compared to wild-type.

FIG. 6: provides data showing that uricase inhibition by oxonic acid OXO) and commensurate increased uric acid is associated with increased total kidney to body weight ratio (2K/BWT) in a rat model of polycystic kidney disease (PCK) and in a mouse model of polycystic kidney disease (Pkd1RC/RC). After 70 days exposure to oxonic acid both models showed similar results, increased of serum uric acid resulted in an increase in 2K/BWT, increased cyst sized and increased serum creatinine (P<0.05; P<0.05; for rat and mouse creatinine P<0.07 & P<0.05). Increased serum uric acid is associated with structural and functional changes in kidney function. Increased creatinine is associated with decreased filtering capacity of kidneys disease.

FIG. 7: provides data showing that in a rodent model of polycystic kidney disease uricase inhibition by oxonic acid and commensurate increased uric acid shows decreased kidney function as indicated by increased creatinine in serum.

FIG. 8: In a rodent model of polycystic kidney disease-mouse RC/RC model-xanthine oxidase inhibition attenuates kidney disease progression as indicated by average total kidney volume (FIG. 8A) and median size of kidney (FIG. 8B)

FIG. 9: is a graph showing that in a rodent model of polycystic kidney disease—mouse RC/RC-oxonic acid (OXO) exposure increased kidney size. When OXO and Xanthine oxidase inhibition (“OXY+L”, oxypurinol & L-arginine) are administered simultaneously, OXY+L attenuated the oxonic acid effect and normalized/decreased uric acid induced kidney expansion at a moderate (8 mg/kg/day), or at a higher oral dose (24 mg/kg/day).

FIG. 10 shows an embodiment of a diagnostic device.

### DETAILED DESCRIPTION

The invention relates to methods of diagnosis, methods treatment and uses of agents affecting aberrant purine metabolism. The invention relates to methods of diagnosing aberrant purine metabolism in a tissue, methods of controlling aberrant purine metabolism in a tissue, and methods of treating aberrant purine metabolism in a tissue.

The invention relates to the use of xanthine oxidase inhibitors in polycystic kidney disease and/or combinations of agents capable of decreasing tissue expression of xanthine oxidase enzyme or xanthine oxidase activity.

Without being bound to a particular thereof, data provided herein suggests that where tissue expression of XO/XOR contributes to intracellular uric acid and oxygen radical production, this can contribute to mitochondrial injury, vascular or structural injury, inflammation or fibrosis. Compositions and methods are provided that ameliorate such detrimental effects.

Treatment of ADPKD to specifically inhibit intracellular, kidney, or other tissue injury with the therapeutic use of a xanthine oxidase inhibitor or, free oxygen radical scavenger or a molecule that has both properties is also provided.

Further, a XOI in combination with an anti-inflammatory or anti-fibrotic drug may be used to protect kidney from progression of disease.

Further a XOI in combination with an “vasopressin receptor antagonist” such as tolvaptan, lixivaptan, or other “-vaptan” to initiation of cysts/growth of cysts or increased total kidney volume as well as slow decline of function and decrease the health consequences of both extracellular and intracellular xanthine oxidase.

Further an effector of xanthine oxidase/xanthine dehydrogenase (XO/XDH) expression or activity or effector of the ratio of XO/XDH such as an SGLT2 inhibitor or Sirtuin 1 activator capable of modulating XO/XDH directly or indirectly, is administered to a subject exhibiting symptoms of PKD.

Further, also disclosed is the silencing of genes, such as xanthine oxidase (XO), thioredoxin-interacting protein (TXNIP), and/or nuclear factor erythroid 2-related factor (nrf-2/heme oxygenase 1(HO-1), that uses interfering molecules directed to expression of such genes to reverse the health consequences of overexpression.

According to certain embodiments, provided is a method of detecting and treating kidney disease progression in a subject. The method involves obtaining a sample from a subject; and detecting a level of xanthine oxidase expression or activity in a sample, and if the level of xanthine oxidase expression or activity is at or above a predetermined level, or is elevated relative to a control, determining the subject is a subject in need; and administering a therapeutically effective amount of xanthine oxidase inhibitor to the subject in need.

Also provided is a method comprising detecting changes to xanthine oxidase (XO) concentration or activity or xanthine dehydrogenate (XDH) concentration or activity, or the XO/XDH ratio of concentration or activity. The method involves obtaining a sample from a subject exhibiting one or more symptoms of polycystic kidney disease, wherein the sample comprises a blood sample or urine sample, or extracellular vesicle sample from the blood sample or the urine sample; detecting XO concentration or activity and XDH concentration or activity in the sample; and administering a UALA and, optionally co-administering an organic base, if the sample comprises XO concentration or activity, XDH concentration or activity, and/or an XO/XDH ratio of concentration or activity that is a deviation from that of a healthy subject. In a specific example, a deviation from that of a healthy subject is a XO concentration above 1 mg/L; XO enzyme activity above 105 (U/L); and/or a XO/XDH that is at least 0.1-10% disparate with that of a healthy subject.

A further method embodiment involves a method of reducing aberrant purine metabolism associated with kidney disease in a subject in need. This embodiment involves administering a therapeutically effective amount of one or more uric acid lowering agents, wherein the method treats a symptom of cystic disease, and wherein the one or more uric acid lowering agents are optionally selected from the group consisting of xanthine oxidase inhibitor and sirtuin-1 activator. In a specific embodiment, administering comprises co-administering a oxypurinol and a gliflozin.

In yet another embodiment, provided is method of reducing markers of kidney disease progression in a subject. The method involves obtaining a sample from the subject; detecting a marker for kidney disease, the marker comprising tissue oxygen radicals, uric acid, cytokines, inflammatory cells, fibrosis, mitochondriosis, or Sirtuin-1 in a sample, and if the level of marker is above baseline, administering a therapeutically effective amount of UALA and, optionally, co-administering an organic base, to the subject in need. In a specific example, the UALA is an interfering molecule that targets xanthine oxidase or xanthine dehydrogenase expression, or is an eRNA that increases expression of sirtuin-1. In a more specific embodiment, administering comprises co-administering a xanthine oxidase inhibitor and an interfering molecule that targets xanthine oxidase or xanthine dehydrogenase expression.

Also provided is a method comprising detecting presence of a marker, or a ratio of markers in a biological sample from a subject; and if the marker is elevated relative to a baseline or control, or if a ratio of markers is disproportionate to a baseline or control, administering a therapeutically effective amount of at least one uric acid lowering agent to the subject. The marker may be one or more of xanthine oxidase, xanthine dehydrogenase, a sirtuin or Sirtuin-1, Hypoxia Inducing Factor-1 (HIF-1), Erythropoietin, PCNA, Wnt/B-Catenin, IL-5, IL-6, STAT1, STAT2, mTOR, TNFa, MIF, NLRP3 Inflammasome, or constituents of blood or urine borne cell membranes, micro-vesicles, apoptotic bodies, exosomes, or free enzymes or specific portions/fragments of enzymes.

Other method embodiments relate to a method of monitoring effectiveness of a treatment of polycystic kidney disease. The method involves administering an amount of a uric acid lowering agent to a subject exhibiting elevated xanthine oxidase activity or concentration or elevated xanthine oxidase/xanthine dehydrogenase activity or concentration ratio in urine in a subject relative to baseline; and detecting xanthine oxidase concentration or activity or xanthine oxidase/xanthine dehydrogenase concentration or activity ratio in urine in a subject, wherein a decrease in xanthine oxidase activity or concentration or a migration of the xanthine oxidase/xanthine dehydrogenase concentration or activity ratio toward baseline indicates effectiveness of treatment. A further method embodiment involves a method of treating polycystic kidney disease that comprises co-administering therapeutically effective amounts of xanthine oxide inhibitor and metformin.

Also disclosed is a composition comprising an amount of a oxypurinol and an amount of metformin, and/or an SGLT2 Inhibitor. In a specific embodiment, the composition comprises oxypurinol, metformin and gliflozin.

In alternative method embodiments, provided is a method of treating polycystic kidney disease that involves co-administering therapeutically effective amounts of xanthine oxide inhibitor and tolvaptan and/or lixivaptan

In a related embodiment, provided is a composition comprising a therapeutically effective amount of a uric acid lowering agent and a therapeutically effective amount of tolvaptan and/or lixivaptan. In a specific example the UALA is a xanthine oxidase inhibitor.

Also disclosed, is a method of treating cystic disease in a subject in need, the method comprising co-administering a therapeutically effective amount of a uric acid lowering agent and gliflozin, wherein gliflozin is co-administered in an amount to increase the aqueous solubility and bioavailability of the uric acid lowering agent.

In a further embodiment, provided is a method of treating a cystic disease that involves co-administering a therapeutically effective amount of a xanthine oxidase inhibitor and a Sirtuin-1 activator. In a specific example, the sirtuin-1 activator is a SGLT2 inhibitor.

In yet another embodiment, provided is a method of treating cystic disease in a subject in need that involves co-administering a therapeutically effective amount of at least one xanthine oxidase inhibitor and an agent that directly or indirectly decreases expression of xanthine oxidase. The agent that directly decreases expression of xanthine oxidase may include an interfering molecule targeting xanthine oxidase expression. The agent that indirectly decreases expression of xanthine oxidase may include a sirtuin-1 activator. In a specific example, the sirtuin-1 activator comprises a gliflozin or an eRNA that induces expression of sirtuin-1, or a combination thereof.

In a related embodiment, provided is a composition that includes a therapeutically effective amount of a xanthine oxidase inhibitor and an agent that directly or indirectly decreases expression of xanthine oxidase. The agent that directly decreases expression of xanthine oxidase may include an interfering molecule targeting xanthine oxidase expression. The agent that indirectly decreases expression of xanthine oxidase may include a sirtuin-1 activator. In a specific embodiment, the sirtuin-1 activator comprises a gliflozin or an eRNA that induces expression of sirtuin-1, or a combination thereof.

### Definitions

The preferred materials and methods are described herein; any methods and materials similar or equivalent to those described herein can be used in the practice of or testing of the invention. Unless defined otherwise, all technical and scientific terms herein have the same meaning as commonly understood by one of ordinary skill in the art to which the invention pertains. In describing and claiming the present invention, the following terminology will be used. It is also to be understood that the terminology used herein is for the purpose of describing particular embodiments only, and it is not intended to be limiting.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

The articles “a,” “an,” “the” and the like are used herein to refer to one or to more than one (i.e., to at least one) of the grammatical object of the article, unless specifically noted otherwise. By way of example, “an element” means one element or more than one element. Unless otherwise indicated, “or” encompasses “and.” To illustrate, “A, B, or C” means A alone, B alone, C alone, the combination of A and B, the combination of A and C, the combination of B and C, and the combination of A, B, and C, unless otherwise illustrated.

The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

The term “aberrant purine metabolism” as used herein refers to a conversion of purines to a metabolite such as xanthine, uric acid (or urate) and/or allantoin that is above levels exhibited in a healthy subject. Aberrant purine metabolism is typically associated with elevated expression and/or activity of xanthine oxidase in cells of a subject exhibiting one or more symptoms of a disease (e.g. polycystic kidney disease, cystic liver disease or cystic disease or any disease where expression of xanthine oxidase in a tissue or activity is increased).

The terms “administering” or “administration” and other grammatical forms thereof refers to any route of introducing or delivering to a subject a compound or agent to perform its intended function. The administering or administration can be carried out by any suitable route, including orally, intranasally, parenterally (intravenously, intramuscularly, intraperitoneally, or subcutaneously), rectally, or topically. Administering or administration includes self-administration and the administration by another.

As used herein, the term “antisense sequence” refers to an oligomeric compound that is at least partially complementary to a target nucleic acid molecule to which it hybridizes. In certain embodiments, an antisense compound modulates (increases or decreases) expression of a target nucleic acid. Antisense compounds include, but are not limited to, compounds that are oligonucleotides, oligonucleosides, oligonucleotide analogs, oligonucleotide mimetics, and chimeric combinations of these.

As used herein, the terms “co-administered, “co-administering,” or “concurrent administration” when used, for example with respect to administration of an exemplary therapeutic agent with another exemplary therapeutic agent, or a conjunctive agent along with administration of an exemplary therapeutic agent refers to administration of the exemplary therapeutic agent and the other exemplary therapeutic agent or conjunctive agent such that both can simultaneously achieve a physiological effect. The two agents, however, need not be administered together. In certain embodiments, administration of one agent can precede administration of the other, however, such co-administering typically results in both agents being simultaneously present in the body (e.g. in the plasma) of the subject.

As used herein, a “composition,” “pharmaceutical composition” or “therapeutic agent” all include a composition comprising at least a uric acid lowering agent. Optionally, the “composition,” “pharmaceutical composition” or “therapeutic agent” further comprises pharmaceutically acceptable diluents or carriers, and/or a conjunctive agent. In the case of an interfering molecule, for example, the interfering molecule may be combined with one or more pharmaceutically acceptable diluents, such as phosphate-buffered saline, for example.

The term “control” when used in the context of describing a comparison with a marker, or ratio of markers, refers to a level of a molecule or ratio of molecules representative of present in samples from a healthy subject.

The term “cystic disease” as used herein refers to any disease that contains tissues where cyst genesis or cyst growth is increased relative to a healthy individual. A cystic disease describes a group of conditions that cause cysts (fluid filled sacs) to form in or around an organ. Cystic growth or expansion may be associated with increased pressure on health tissue surrounding the cyst resulting in decreased blood circulation, exchange of nutrients or metabolic products that modulates disease progression. Expression of xanthine oxidase/xanthine dehydrogenase (XO/XDH) or the ratio of XO/XDH in a cystic tissue may result in the direct accumulatio of uric acid in a tissue, or oxygen radical accumulation in a tissue that directly or indirectly promotes disease or disease progression in a tissue.

The term “cystic kidney disease” Cystic kidney disease causes cysts (sacs of fluid) to form in or around the kidneys. There are many types of cystic kidney disease. Some are the result of abnormal genes; others develop in the womb or as a result of kidney failure. Both adults and infants can have cystic kidney disease. The term cystic kidney disease includes polycystic kidney disease.

Enhancer RNAs, or eRNAs, as used herein refer to short non-coding RNA molecules that are transcribed from the loci of enhancers. Administration of eRNAs relating to an enhancer of a target gene can increase expression of a target gene. Enhancers are regulating elements in the genome that cooperate with promoters to control target gene transcription and cell fate alongside promoters. Promoters of genes such as uricase or sirtuin may be particularly useful for modulating disease progression. Sequences of human sirtuin 1 are found at accession numbers NM_012238, NM_001142498, and NM_001314049.

As used herein, “extracellular vesicles” encompasses “exosomes,” or “microvesicles (MVs),” which are released by almost all types of cells upon fusion of its multi-vesicular body with a plasma membrane of the cell, in some embodiments. The term “extracellular vesicles” may include both exosomes and MVs. Extracellular vesicles are present in many, if not all, eukaryotic fluids, including blood, urine and cultured medium of cell cultures. Extracellular vesicles, in particular exosomes or MVs, are known for their role in cell to cell communications and have demonstrated an ability to unload their contents and contribute to the transformation of normal and stem cells to cancerous states. Microvesicles, for example, can be formed by a variety of processes, including the release of apoptotic bodies, the budding of microvesicles directly from the cytoplasmic membrane of a cell, and exocytosis from multivesicular bodies. For example, extracellular vesicles are commonly formed by their secretion from the endosomal membrane compartments of cells as a consequence of the fusion of multivesicular bodies with the plasma membrane. The multivesicular bodies (MVBs) are formed by inward budding from the endosomal membrane and subsequent pinching off of small vesicles into the luminal space. The internal vesicles present in the MVBs are then released into the extracellular fluid as so-called exosomes or extracellular vesicles.

The term “healthy subject” is a subject that lacks symptoms of a disease or genetic predisposition to the disease. In a specific example, a healthy subject does not exhibit symptoms of polycystic kidney disease, and/or lacks genetic predisposition for PKD.

The term “inhibitory oligonucleotide” refers to any oligonucleotide that reduces the production or expression of proteins, such as by interfering with translating mRNA into proteins in a ribosome or that are sufficiently complementary to either a gene or an mRNA encoding one or more of targeted proteins, that specifically bind to (hybridize with) the one or more targeted genes or mRNA thereby reducing expression or biological activity of the target protein. Inhibitory oligonucleotides include isolated or synthetic shRNA or DNA, siRNA or DNA, antisense RNA or DNA, Chimeric Antisense DNA or RNA, miRNA, and miRNA mimics, among others.

As used herein, the terms “interfering molecule” refer to all molecules that have a direct or indirect influence on gene expression, such as the silencing of a target gene sequence. Interfering molecules include inhibitory oligonucleotides, RNA interfering molecules and RNA-like interfering molecules. Examples of interfering RNA molecules include antisense sequences, siRNAs, short hairpin RNAs (shRNAs), single-stranded siRNAs, microRNAs (miRNAs), methylated siRNAs or other siRNAs treated to protect the siRNA from degradation by circulating Rnases, and dicer-substrate 27-mer duplexes. Examples of “RNA-like” molecules include, but are not limited to, siRNA, single-stranded siRNA, microRNA, and shRNA molecules that contain one or more chemically modified nucleotides, one or more non-nucleotides, one or more deoxyribonucleotides, and one or more non-phosphodiester linkages. Thus, siRNAs, single-stranded siRNAs, shRNAs, miRNAs, and dicer-substrate 27-mer duplexes are subsets of “interfering molecules. “Interfering molecules” also may include PMOs. See US Pat Pub. 20200299698, Chinese Patent CN104232644; or Wang et al. Oxid Med Cell Longev, 2022, 2022:4326695, and Origene Technologies (Cat No. TF308350).

The term “marker” or “biomarker” as used herein refers to a biological molecule whose presence or relative presence compared to baseline or control is indicative of a disease or disorder. In the context of the present disclosure, examples of markers include xanthine oxidase and/or xanthine dehydrogenase (or nucleic acid sequences encoding same or fragments thereof), antigens associated with xanthine oxidase and/or xanthine dehydrogenase, and/or uric acid, and/or genetic markers for PKD.

The term “metformin” refers to metformin or a pharmaceutically acceptable salt thereof. See U.S. Pat. No. 6,031,004, RE46,496, and U.S. Pat. No. 10,154,972.

The term micro-RNA (abbreviated miRNA) is a small non-coding RNA molecule (containing about 22 nucleotides) found in plants, animals and some viruses, that functions in RNA silencing and post-transcriptional regulation of gene expression. The miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA. Under a standard nomenclature system, names are assigned to experimentally confirmed miRNAs. The prefix “miR” is followed by a dash and a number, the latter often indicating order of naming. “MIR” refers to the gene that encodes a corresponding miRNA. Different miRNAs with nearly identical sequences except for one or two nucleotides are annotated with an additional lower case letter. The term miRNA mimics, refers to small, double-stranded RNA molecules, such as siRNA, designed to mimic endogenous mature miRNA molecules when introduced into cells.

The term “organic base” as used herein refers to the organic base may be (a) a Class 1 base with a pKa1 between about 7 to 13 including but not limited to L-arginine, D-arginine, choline, L-lysine, D-lysine, and caffeine. In an alternative embodiment, the organic base is a biguanide (U.S. Pat. No. 9,480,663) or biguanidine such as metformin, phenformin, buformin or a salts thereof. It is noted that metformin has multiple beneficial effects in the context of compositions and methods described herein. First, it can act as a solubilizing agent for oxypurinol to increase oral bioavailablity. Secondarily, it can act as an alkalinizing agent. Thirdly, metformin here can act as a uric acid solubilizing agent. An unexpected result of metformin has been found that a 1:01 to 1:10 ratio of oxypurinol to metformin increases oxypurinol solubility from 0.2 mg/ml to 16.8 mg/ml, or more. The gliflozin family of SGLT2 inhibitors are also basic and can serve as an organic base in accord with the teachings herein.

As used herein, the terms “phosphothioate morpholino oligomer(s),” “a PMO” or “PMOs” refer to molecules having the same nucleic acid bases naturally found in RNA or DNA (i.e. adenine, cytosine, guanine, uracil or thymine), however, they are bound to morpholine rings instead of the ribose rings used by RNA. They may also linked through phosphorodiamidate rather than phosphodiester or phosphorothioate groups. This linkage modification eliminates ionization in the usual physiological pH range, so PMOs in organisms or cells are uncharged molecules. The entire backbone of a PMO is made from these modified subunits.

The term “polycystic kidney disease” or “PKD” as used herein refers to a genetic disorder in which the renal tubules become structurally abnormal, resulting in the development and growth of multiple cysts within the kidney. These cysts may begin to develop in utero, in infancy, in childhood, or in adulthood. Cysts are non-functioning tubules filled with accumulated fluid within them, which range in size from microscopic to enormous, crushing or compressing adjacent normal tubules, and/or healthy adjacent tissues and blood vessels and eventually rendering them non-functional as well.

As used herein, the term “prevent”, “prevention” or “preventing” means causing the clinical symptoms of the disease state not to develop, e.g., inhibiting the onset of disease, in a subject that may be exposed to or predisposed to the disease state, but does not yet experience or display symptoms of the disease state. The terms as used herein may further include either 1) the reduction in frequency or severity of symptoms commonly associated with the disorder; or 2) a delay or avoidance of additional symptoms associated with the condition or disease, or complete prevention of the disease. One skilled in the art will recognize that wherein the various embodiments are directed to methods of prevention, a subject in need thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

As used herein, the term SGLT2 inhibitor refers to sodium-glucose co-transporter inhibitors. A non-limiting list of SGLT2 inhibitors includes, but is not limited to, “gliflozin(s)” such as empagliflozin, dapagliflozin, canagliflozin, ertugliflozin, Ipragliflozin, Luseogliflozin, Remogliflozin, Sergliflozin, Sotagliflozin or Tofogliflozin. It has been discovered that the gliflozins possess multi-functional properties that are particularly beneficial in treating cystic kidney disease. Gliflozins can reduce expression of xanthine oxidase as a function of activating sirtuin-1, increase bioavailability of other uric acid lowering agents such as xanthine oxidase inhibitors as a function of their basic nature, and can themselves inhibit xanthine oxidase activity. Thus, gliflozins fall within the definitions of uric acid lowering agent, xanthine oxidase inhibitor, sirtuin-1 activator, and organic base. To the extent that gliflozins are described or claimed in combination with or co-administered with one or more of these four types of agents, this is intended to convey that reference to gliflozin in that context may serve an additional function relative to the function of the agent to which it is combined with or co-administered with unless stated otherwise. For example, a composition comprising a UALA and a gliflozin, means that the gliflozin may serve as a UALA and something other than a UALA, such as an organic base to increase bioavailability of a UALA.

As used herein, “shRNAs” (small hairpin RNAs) are short “hairpin-turned” RNA sequences that may be used to inhibit or suppress gene expression.

As used herein, “siRNAs” (short interfering RNAs), also known as small interfering RNA or silencing RNA refer to double-stranded RNA molecules, generally around 15-30 nucleotides in length, that are complementary to the sequence of the mRNA molecule transcribed from a target gene, and interferes with the expression of the target gene.

As used herein, the term “sirtuin-1 activator” refers to an agent capable of activating the sirtuin family of NAD+-dependent protein lysing deacetylases and modulating the various function of sirtuins, such as physiological, metabolic and stress responses. More specifically, agents that activate sirtuin-1 (SIRT-1) and thereby modulate expression of xanthine oxidase enzyme, xanthine dehydrogenase enzyme or modulate the ratio of XO/XDH in a tissue. Examples of agents capable of activating sirtuins include resveratrol, epicatechin, quercetin, SRT2104, SRT1720, 1,4-DHP derivate, UBCS039, SRT2104, SRT2379, SRT3025, or SGLT-2 inhibitors including “gliflozins” such as empagliflozin, dapagliflozin, canagliflozin an eRNA that induces expression of sirtuin-1. Other examples of sirtuin-1 activators are described in US Pat. Pub. 20070149466.

As used herein, the term “subject” or “patient” refers to any animal (e.g., a mammal), including, but not limited to, humans, non-human primates, rodents, and the like, which is to be the recipient of a particular treatment.

As used herein, a “subject in need” is a subject who exhibits one or more symptoms of PKD and/or is genetically pre-disposed to developing PKD. Symptoms of PKD can include increased kidney size, reduced kidney function, increased rate cyst formation, increased rate of cyst growth, increased high blood pressure, increased abdominal or back pain, increased cardiovascular disease, increased rate of kidney stone formation, increased incidence of gout, increased incidence of kidney failure, increased endothelial dysfunction, increased inflammatory state, increased rheumatoid arthritis, increased urinary tract infection, increase incidence of heart attack or stroke, increased need for dialysis, increased circulating creatinine concentration or serum uric acid concentration greater than 6 mg/dL.

A “therapeutically effective amount” refers to an amount which, when administered in a proper dosing regimen, is sufficient to reduce or ameliorate the severity, duration, or progression of the disorder being treated (e.g., cancer), prevent the advancement of the disorder being treated (e.g., cancer), cause the regression of the disorder being treated (e.g., cancer), or enhance or improve the prophylactic or therapeutic effects(s) of another therapy. The full therapeutic effect does not necessarily occur by administration of one dose and may occur only after administration of a series of doses. Thus, a therapeutically effective amount may be administered in one or more administrations per day for successive days.

As used herein, the terms “treat” “treating”, “treatment” or “alleviation” refers to therapeutic treatment, wherein the object is to halt, slow down or reverse a pathological condition or disorder In one example, the terms may further include administering a compound to manage the symptoms or underlying cause of a condition with the goal of reducing symptoms or signs of the disease and either to prevent or to slow progression, to arrest or potentially to reverse manifestations of the disease, or to inhibit the underlying mechanism(s) causing the disease.

The term “uric acid lowering agent” or UALA refers to substances known to lower serum uric acid levels in mammals. Typically, the UALA may limit serum uric acid levels by at least about 0.2 mg/dl. UALAs include, but are not limited to, xanthine oxidase inhibitors; uricosurics such as benziodarone, benzbromarone, probenecid; uricase derivatives such as Rasburicase and Pegylated uricase; gene based therapies such as uricase overexpression or blockade of URAT-1; a supplement of the uricase protein which might be delivered as a conjugate with polyethylene glycol or another delivery system; an interfering molecule that targets xanthine oxidase or xanthine dehydrogenase, a sirtuin-1 activator such as an SGLT-2 inhibitor (e.g. gliflozin), a flavonoid such as reseveratrol, or an eRNA that upregulates expression of sirtuin-1; and a urate channel inhibitor. As is described above, gliflozins possess multiple mechanistic functions/

The term “xanthine oxidase inhibitor” as used herein refers to an agent that reduces the activity or expression of xanthine oxidase. Examples of xanthine oxidase inhibitors include, but are not limited to, allopurinol, oxypurinol, hydroxyakalone, TEI-6720, carprofen, febuxostat, topiroxostat, TMX-049 and y-700; inhibitory oligonucleotide or other interfering molecules targeting xanthine oxidase expression; or anti-xanthine oxidase antibodies. A xanthine oxidase inhibitor may be further defined by its ability or the ability of a metabolic product of a prodrug to separately or simultaneously inhibit xanthine oxidase or xanthine dehydrogenate in blood, tissue or fluid or all.

The compounds are preferably formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that reduces serum uric acid levels at least 0.5 mg/dl to be equal to or less than 5.5 mg/dl. In a most preferred embodiment, effective amount is such as to lower serum uric acid levels to less than or equal to 5.5 mg/dl and more than or equal to 4.0 mg/dl. Preferably still, the effective amount is such as to lower serum uric acid levels to less than or equal to 5.2 mg/dl and more than or equal to 4.5 mg/dl.

The UALA may be administered concomitantly or sequentially (i.e., co-administered) with one or more known antioxidants, such as, but not limited to, vitamin C, alpha-lipoic acid, Vitamin E, beta carotene, selenium, zinc, carnosine, green tea, soy and isoflavones, tempol, etc. In other embodiments, the uric acid lowering agent is combined with a vasopressin receptor antagonist, or SGLT-2 inhibitor or agents capable of increasing aqueous solubility of the xanthine oxidase inhibitor or its bioavailablity. Such combination may be beneficial regardless of uric acid levels, but may be particularly helpful if dosages of UALA are administered that lower the uric acid below 5.5 mg/dl. In other embodiments, the uric acid lowering agent is co-administered with metformin. Accordingly, in another embodiment, provided is a composition that includes both a uric acid lowering agent and metformin.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

### In Vitro Diagnostic Device

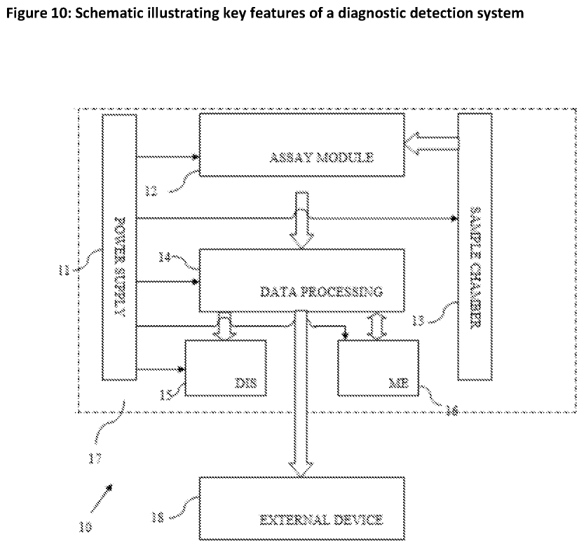

FIG. 10 schematically illustrates an inventive in vitro diagnostic device shown generally at 10. An inventive in vitro diagnostic device includes at least a sample collection chamber 13, an assay module 12 used to detect biomarkers of disease, and a user interface that relates the concentration (level) of the measured biomarker measured in the assay module. The in vitro diagnostic device may be a handheld device, a bench top device, or a point of care device.

The sample chamber 13 can be of any sample collection apparatus known in the art for holding a biological fluid. In one embodiment, the sample collection chamber can accommodate any one of the biological fluids herein contemplated, such as whole blood, cells, cell suspension, cell or tissue lysates, plasma, serum, urine, sweat or saliva.

The assay module 12 is preferably made of an assay which may be used for detecting a protein in a biological sample, for instance, through the use of antibodies in an immunoassay. The assay module 12 may include any assay currently known in the art; however, the assay should be optimized for the detection of a marker used for diagnosing disease (e.g., polycystic kidney disease), severity of injury, or responsiveness to therapy in a subject. The assay module 12 is in fluid communication with the sample collection chamber 13. In one embodiment, the assay module 12 is configured to conduct an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. Alternatively, the assay module is configured to conduct a nucleic acid hybridization assay. In one embodiment a colorimetric assay may be used which may include only of a sample collection chamber 13 and an assay module 12 of the assay. Although not specifically shown these components are preferably housed in one assembly 17.

In one embodiment, the inventive in vitro diagnostic device contains a power supply 11, an assay module 12, a sample chamber 13, and a data processing module 14. The power supply 11 is electrically connected to the assay module and the data processing module 14. The assay module 12 and the data processing module 14 are in electrical communication with each other. As described above, the assay module 12 may include any assay currently known in the art; however, the assay should be optimized for the detection of the biomarkers used herein for detecting injury disease, or repair in a subject. The assay module 12 is in fluid communication with the sample collection chamber 13. The assay module 12 includes of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. A biological sample is placed in the sample chamber 13 and assayed by the assay module 12 detecting for a marker. The measured amount of the marker by the assay module 12 is then electrically communicated to the data processing module 14. The data processing 14 module may include any known data processing element known in the art, and may include a chip, a central processing unit (CPU), or a software package which processes the information supplied from the assay module 12.

In one embodiment, the data processing module 14 is in electrical communication with a display 15, a memory device 16, or an external device 18 or software package [such as laboratory and information management software (LIMS)].

In one embodiment, the data processing module 14 is used to process the data into a user defined usable format. This format includes the measured concentration (levels) of one or more markers detected in the sample, and that are useful for diagnosing a disease (such as polycystic kidney disease), severity of injury, or responsiveness to therapy in a subject. The information from the data processing module 14 may be illustrated on the display 15, saved in machine readable format to a memory device, or electrically communicated to an external device 18 for additional processing or display. Although not specifically shown these components are preferably housed in one assembly 17. In one embodiment, the data processing module 14 may be programmed to compare the detected amount of the biomarker transmitted from the assay module 12, to a comparator algorithm. The comparator algorithm may compare the measured amount to the user defined threshold which may be any limit useful by the user. In one embodiment, the user defined threshold is set to the amount of the biomarker measured in control subject, or a statistically significant average of a control population.

In one embodiment, an in vitro diagnostic device may include one or more devices, tools, and equipment configured to hold or collect a biological sample from an individual. In one embodiment of an in vitro diagnostic device, tools to collect a biological sample may include one or more of a needle, swab, a scalpel, a syringe, a scraper, a container, and other devices and reagents designed to facilitate the collection, storage, and transport of a biological sample. In one embodiment, an in vitro diagnostic test may include reagents or solutions for collecting, stabilizing, storing, and processing a biological sample. These reagents include antibodies, aptamers, or combinations thereof raised against one of the aforementioned biomarkers. In one embodiment, an in vitro diagnostic device, as disclosed herein, may include a micro array apparatus and reagents, and additional hardware and software necessary to assay a sample to detect and visualize the temporally relevant biomarkers.

### Kits

In yet another aspect, disclosed are kits for aiding a diagnosis of injury, disease, or repair, including type, phase amplitude (severity), subcellular localization, wherein the kits may be used to detect the markers. For example, the kits can be used to detect any one or more of the biomarkers described herein, which markers are differentially present in samples of a patient and normal subjects. In another example, the kits can be used to identify compounds that modulate expression of one or more of the markers in in vitro or in vivo animal models to determine the effects of treatment.

In one embodiment, a kit includes (a) an antibody, aptamer, or nucleic acid probe that specifically binds to an aforementioned marker; and (b) a detection reagent. Such kits are prepared from the materials described above, and the previous discussion regarding the materials (e.g., antibodies, aptamers detection reagents, immobilized supports, etc.) being fully applicable to this section and thus is not repeated. [0086] In one inventive embodiment, the kit includes (a) a composition of detecting agent to detect one or more markers.

In one embodiment, the invention includes a diagnostic kit for use in screening biological samples for presence or differential amounts of xanthine oxidase and/or xanthine dehydrogenase and/or uric acid and/or sirtuin-1. The diagnostic kit in this embodiment includes a substantially isolated antibody or aptamer specifically immunoreactive with peptide or polynucleotide antigens, or nucleic acid probes that hybridize with polynucleotide biomarkers, and visually detectable labels associated with the binding of the polynucleotide or peptide antigen to the antibody or aptamer or nucleic acid probe. In one embodiment, the antibody or aptamer is attached to a solid support. Antibodies or aptamers used in the inventive kit are those raised against any one of the biomarkers used herein for temporal data. In one embodiment, the antibody is a monoclonal or polyclonal antibody or aptamer raised against the rat, rabbit or human forms of the biomarker. The detection reagent of the kit includes a second, labeled monoclonal or polyclonal antibody or aptamer. Alternatively, or in addition thereto, the detection reagent includes a labeled, competing antigen.

In one diagnostic configuration, test serum is reacted with a solid phase reagent having a surface-bound antigen obtained by the methods of the present invention. After binding with specific antigen antibody or aptamer to the reagent, the reagent is reacted with reporter-labeled anti-human antibody or aptamer to bind reporter to the reagent in proportion to the amount of bound anti-antigen antibody or aptamer on the solid support. The reagent is again washed to remove unbound labeled antibody or aptamer, and the amount of reporter associated with the reagent is determined. Typically, the reporter is an enzyme which is detected by incubating the solid phase in the presence of a suitable fluorometric, luminescent or colorimetric substrate.

The solid surface reagent in the above assay is prepared by known techniques for attaching protein or oligonucleotide material to solid support material, such as polymeric beads, dip sticks, a well (96-well plate) or filter material. These attachment methods generally include non-specific adsorption of the protein oligonucleotide to the support or covalent attachment of the protein or oligonucleotide, typically through a free amine group, to a chemically reactive group on the solid support, such as an activated carboxyl, hydroxyl, or aldehyde group. Alternatively, streptavidin coated plates can be used in conjunction with biotinylated antigen(s).

In another embodiment, the kit may include materials for polymerase chain reaction PCR expansion to facilitate detection of genetic material, prior to testing the amount of marker detected for a diagnosis of injury, disease or repair, including type, phase, amplitude (severity), subcellular localization, disease state and or effect of treatment on a patient.

In some embodiments, the kit may include a standard or control information so that the test sample can be compared with the control information standard to determine if the test amount of a marker detected in a sample is a diagnostic amount consistent with a diagnosis of injury, disease, or repair, including type, phase, amplitude (severity), subcellular localization, disease state and/or effect of treatment on the patient.

In one embodiment, a kit includes: (a) a substrate including an adsorbent thereon, wherein the adsorbent is suitable for binding a marker (e.g. xanthine oxidase and/or xanthine dehydrogenase, and/or uric acid), and optionally, (b) instructions to detect the marker by contacting a sample with the adsorbent and detecting the marker retained by the adsorbent. In some embodiments, the kit may include an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In certain embodiments, the kit further includes instructions for suitable operational parameters in the form of a label or a separate insert. For example, the kit may have standard instructions informing a consumer how to wash the probe after a sample is contacted on the probe. In another example, the kit may have instructions for pre-fractionating a sample to reduce complexity of proteins in the sample. In another example, the kit may have instructions for automating the fractionation or other processes.

### Biological Samples

The methods of detection provide the ability to detect and monitor levels of xanthine oxidase and/or xanthine dehydrogenase, and/or uric acid which are present biological samples provide enhanced diagnostic capability by allowing clinicians to determine the presence, phase and amplitude (severity) of disease. A biological sample operative herein includes body fluids such as blood, plasma, serum, tears, perspiration, urine, fecal matter, cells, tissues, cell or tissue lysates, whole blood, or other biological samples recognized in the art to obtain evidence of increased uric acid concentration or increase XO expression.

Baseline levels of markers (e.g. XO, xanthine dehydrogenase, (or nucleic acid sequences of same or portions thereof) or uric acid) are those levels obtained in the target biological sample in the species of desired subject in the absence of a known injury, disease, or repair. These levels need not be expressed in hard concentrations but may instead be known from parallel control experiments and expressed in terms of fluorescent units, density units, and the like. Typically, baselines are determined from subjects where there is an absence of a biomarker or present in biological samples at a negligible amount. However, some proteins may be expressed less in an injured, diseased or repaired patient or before any clinical measures of injury, disease, or repair. Determining the baseline levels of protein biomarkers in a particular species is well within the skill of the art. Typically, elevated levels of xanthine oxidase or uric acid above baseline, or having a higher ratio of xanthine oxidase to xanthine dehydrogenase, or otherwise deviates with that compared to samples of healthy subjects is indicative of disease and can be compared over time or quantified to determine disease state, or severity of disease, or to determine effectiveness of treatment.

Markers associated with a tissue containing a cyst are those obtained in the target biological sample in the species of desired subject in the absence of a known injury, disease, or repair. Levels of markers co-locating with xanthine oxidase/dehydrogenase expression or activity may be associate with the cystic tissue and directly or indirectly modulated by xanthine oxidase. The marker may be detectable within the circulatory system, lymph system or excretory system and/or associated tissues. Examples of baseline markers include but are not limited to, a sirtuin or Sirtuin-1, Hypoxia Inducing Factor-1 (HIF-1), Erythropoietin, PCNA, Wnt/B-Catenin, IL-5, IL-6, STAT1, STAT2, mTOR, TNFa, MIF, NLRP3 Inflammasome, or constituents of blood or urine borne cell membranes, micro-vesicles, apoptotic bodies, exosomes, or free enzymes or specific portions/fragments of enzymes.

To provide correlations between an injury, disease, or repair and measured quantities of the xanthine oxidase, o, biological samples are collected from subjects in need of measurement for these biomarkers to assess injury, disease, or repair.

The detection methods may be implemented into assays or into kits for performing assays. These kits or assays may alternatively be packaged into a cartridge to be used with an inventive in vitro diagnostic device. Such a device makes use of these cartridges, kits, or assay in an assay module 12, which may be one of many types of assays.

Further, markers such as xanthine oxidase, xanthine dehydrogenase or uric acid can be detected in a biological sample by a variety of conventional methods. For example, immunoassays, include but are not limited to competitive and non-competitive assay systems using techniques such as western blots, radioimmunoassay, ELISA (enzyme linked immunosorbent assay), “sandwich” immunoassays, magnetic immunoassays, radioisotope immunoassay, fluorescent immunoassays, immunoprecipitation assays, precipitin reactions, gel diffusion precipitin reactions, immunodiffusion assays, fluorescent immunoassays, chemiluminescent immunoassays, phosphorescent immunoassays, anodic stripping voltammetry immunoassay, and the like. Inventive in vitro diagnostic devices may also include any known devices currently available that utilize ion-selective electrode potentiometry, microfluids technology, fluorescence or chemiluminescence, or reflection technology that optically interprets color changes on a protein test strip. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation). It should be appreciated, that at present, none of the existing technologies present a method of detecting or measuring any of the ailments disclosed herein, nor does there exist any methods of using such in vitro diagnostic devices to detect any of the disclosed biomarkers to detect their associated injuries.

An exemplary process for detecting the presence or absence of a marker, or relative levels above baseline, alone or in combination, in a biological sample involves obtaining a biological sample from a subject, such as a human, contacting the biological sample with a compound or an agent capable of detecting of the marker being analyzed, illustratively including an antibody or aptamer, and analyzing binding of the compound or agent to the sample after washing. Those samples having specifically bound compound or agent express the marker being analyzed.

In vitro techniques for detection of a marker illustratively include enzyme linked immunosorbent assays (ELISAs), radioimmunoassay, radioassay, western blot, Southern blot, northern blot, immunoprecipitation, immunofluorescence, mass spectrometry, RT-PCR, PCR, liquid chromatography, high performance liquid chromatography, enzyme activity assay, cellular assay, positron emission tomography, mass spectroscopy, combinations thereof, or other technique known in the art.

Examples of enzyme (xanthine oxidase) activity assays include but are not limited to, Cat #8458ScienCell Research Laboratories, Cat #MAK078 by Sigma-aldrich, product no. (ab102522 from Abcam; cat no. EXOX-100 by BioAssay Systems. Another example of a xanthine oxidase activity assay is provided in Atlante A, Valenti D, Gagliardi S, Passarella S. A sensitive method to assay the xanthine oxidase activity in primary cultures of cerebellar granule cells. Brain Res Brain Res Protoc. 2000 November; 6 (1-2):1-5. doi: 10.1016/s1385-299x(00)00030-1. PMID: 11086257. All of the foregoing are incorporated by reference.

Furthermore, in vivo techniques for detection of a marker include introducing a labeled agent that specifically binds the marker into a biological sample or test subject. For example, the agent can be labeled with a radioactive marker whose presence and location in a biological sample or test subject can be detected by standard imaging techniques. It is appreciated that a bound agent assay is readily formed with the agents bound with spatial overlap, with detection occurring through discernibly different detection of each of xanthine oxidase and/or xanthine dehydrogenase and/or uric acid. A color intensity-based quantification of each of the spatially overlapping bound biomarkers is representative of such techniques.

A preferred agent for detecting a marker in a biosample is an antibody, aptamer or nucleic acid probe sequence capable of binding to the biomarker being analyzed. More preferably, the antibody, aptamer or nucleic acid probe sequence is conjugated with a detectable label. Such antibodies can be polyclonal or monoclonal. An intact antibody, a fragment thereof (e.g., Fab or F(ab′)2), or an engineered variant thereof (e.g., sFv) or an aptamer or bi-/tri-specific aptamer can also be used. Such antibodies can be of any immunoglobulin class including IgG, IgM, IgE, IgA, IgD and any subclass thereof. Antibodies and aptamers for numerous inventive biomarkers are available from vendors known to one of skill in the art. Exemplary antibodies operative herein are used to detect a biomarker of the disclosed conditions. In addition, antigens to detect autoantibodies may also be used to detect late injury of the stated injuries and disorders.

An antibody or aptamer is labeled in some inventive embodiments. A person of ordinary skill in the art recognizes numerous labels operable herein. Labels illustratively include, fluorescent labels, biotin, peroxidase, radionucleotides, or other label known in the art. Alternatively, a detection species of another antibody or aptamer or other compound known to the art is used as form detection of a biomarker bound by an antibody or aptamer.

Antibody- and aptamer-based assays operative herein include western blotting immunosorbent assays (e.g., ELISA and RIA) and immunoprecipitation assays. As one example, the biological sample or a portion thereof is immobilized on a substrate, such as a membrane made of nitrocellulose or PVDF; or a rigid substrate made of polystyrene or other plastic polymer such as a microtiter plate, and the substrate is contacted with an antibody or aptamer that specifically binds to a marker under conditions that allow binding of antibody or aptamer to the biomarker being analyzed. After washing, the presence of the antibody or aptamer on the substrate indicates that the sample contained the marker being assessed. If the antibody or aptamer is directly conjugated with a detectable label, such as an enzyme, fluorophore, or radioisotope, the presence of the label is optionally detected by examining the substrate for the detectable label. Alternatively, a detectably labeled secondary antibody or aptamer that binds the marker-specific antibody or aptamer is added to the substrate. The presence of detectable label on the substrate after washing indicates that the sample contained the biomarker.

Numerous permutations of these basic immunoassays are also operative in the invention. These include the biomarker-specific antibody or aptamer, as opposed to the sample being immobilized on a substrate, and the substrate is contacted with a biomarker conjugated with a detectable label under conditions that cause binding of antibody or aptamer to the labeled marker. The substrate is then contacted with a sample under conditions that allow binding of the marker being analyzed to the antibody or aptamer. A reduction in the amount of detectable label on the substrate after washing indicates that the sample contained the marker.

Although antibodies or aptamers are preferred for use in the invention because of their extensive characterization, any other suitable agent (e.g., a peptide or a small organic molecule) that specifically binds a biomarker is operative herein in place of the antibody or aptamer in the above described immunoassays. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

A myriad of detectable labels that are operative in a diagnostic assay for biomarker expression are known in the art. Agents used in methods for detecting a biomarker are conjugated to a detectable label, e.g., an enzyme such as horseradish peroxidase. Agents labeled with horseradish peroxidase may be detected by adding an appropriate substrate that produces a color change in the presence of horseradish peroxidase. Several other detectable labels that may be used are known. Common examples of these detectable labels include alkaline phosphatase, horseradish peroxidase, fluorescent compounds, luminescent compounds, colloidal gold, magnetic particles, biotin, radioisotopes, and other enzymes. It is appreciated that a primary/secondary antibody or aptamer system is optionally used to detect one or more biomarkers. A primary antibody or aptamer that specifically recognizes one or more biomarkers is exposed to a biological sample that may contain the biomarker of interest. A secondary antibody or aptamer with an appropriate label that recognizes the species or isotype of the primary antibody or aptamer is then contacted with the sample such that specific detection of the one or more biomarkers in the sample is achieved.

The present invention provides a step of comparing the quantity of one or more markers to normal levels to determine the disease or disorder of the subject. The results of such a test can help a physician determine whether the administration of a particular therapeutic or treatment regimen may be effective and provide a rapid clinical intervention to the injury or disorder to enhance a patient's recovery.

It is appreciated that other reagents such as assay grade water, buffering agents, membranes, assay plates, secondary antibodies or aptamers, salts, and other ancillary reagents are available from vendors known to those of skill in the art.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

### Compositions and Formulations

The invention provides compositions comprising a UALA and an organic base that enhances or effects solubilisation of the UALA or uric acid or urate in vivo, and optionally a pharmaceutically acceptable carrier, excipient, vehicle or diluent. A xanthine oxidase inhibitor and organic base are preferably selected to ensure maximum solubility, bioavailability or activity of the xanthine oxidase inhibitor without increasing any side effects. Compositions of the invention especially include liquid compositions (e.g. solutions, syrups, colloids, or emulsions). Further, compositions of the invention contemplate micronized, lyophilized or dry-spray powders composed of XOI combined with one or more, organic molecules or organic bases or basic amino acid, that enhance the equilibrium solubility, dissolution or solubility, or bioavailability in an aqueous solution compared to oxypurinol free acid in water. Compositions comprising different combinations of UALAs, sirtuin-1 activators, organic bases and/or conjunctive agents are also contemplated herein.

The invention contemplates a pharmaceutical composition comprising a unit dosage of at least one UALA (e.g. XOI) and an organic base together with a pharmaceutically acceptable carrier, excipient, vehicle, or diluent. A “unit dosage” refers to a unitary i.e. single dose, which comprises all the components of a composition of the invention, which is capable of being administered to a patient. A “unit dosage” may be readily handled and packed, remaining as a physically and chemically stable unit dose comprising the active agent and organic base with pharmaceutical carriers, excipients, vehicles, or diluents. In the alternative, a dosage form kit comprising a xanthine oxidase inhibitor and organic base, and the remaining components, are provided in separate containers, and the inhibitor and base, and remaining components, are combined prior to administration. In particular, a dosage form kit comprises a xanthine oxidase inhibitor and organic base in separate containers, and a solution for use is prepared by combining the ingredients with a suitable carrier, such as sterile water, prior to administration. An unit dose of UALA or xanthine oxidase inhibitor would be a dose sufficient to increase circulating or tissue concentrations of UALA and thereby decrease serum uric acid levels by 1%, 3%, 10%, 30%, 50%, 70%, 90%, 95%, or more and or inhibit tissue xanthine oxidase activity by 1%, 3%, 10%, 30%, 50%, 70%, 90%, 95%, or more, or both simultaneously. Further a dose of UALA would contain an agent with 1, 3, 5, 10, 30, 50, 100, 300, 500, 1000, in units of nanograms, or micrograms or milligrams. More specifically, a unit dose would contain a ratio of UALA to organic base 1:0.01, 1:0.1, 1:1: 1:2, 1:3, 1:5 or 1:10 to increase aqueous solubility and/or oral bioavailablity of the UALA.

According to another embodiment, compositions are provided comprising a XOI, an organic base and/or choline, and an antioxidant. An antioxidant used for composition embodiments may include, but are not limited to, alpha lipoic acid, n-acetylcysteine, vitamin C.

Another embodiment relates to sterile dosage forms of a composition including a UALA and an organic base. In a specific embodiment, the composition comprises a XOI in combination with a biguanidine. In a specific embodiment, a composition is provided that includes an XOI (e.g. oxypurinol, febuxostat, topoxirostat, or allopurinol) and an organic base. The composition may comprise dosage unit that comprises 1-2000 mg of XOI, or 50-2000 mg XOI. In an even more specific embodiment, the dosage unit comprises about 1:1 to about 1:10 molar or weight to weight ratio of oxypurinol to organic base.

A formulation can be provided in a lyophilized form suitable for reconstitution and administration in a subject. Also provided is a formulation where the XOI, organic base, and other ingredients of the composition are provided in a non-lyophilized or lyophilized form separate from each other. The ingredients can be reconstituted and/or solubilized in a suitable sterile liquid and combined to produce a pharmaceutical composition which is suitable for administration to a subject.

The beneficial effects may also be illustrated by increased serum levels of the active ingredients after administration as compared to the active ingredients alone. They may also be demonstrated by a decrease in serum uric acid levels. They may also be demonstrated by increased uric acid solubility, or increased nitric oxide bioavailability or decreased oxygen radical production in an animal, or specifically in man. They may also show decreased xanthine oxidase activity.

A composition of the invention can have increased bioavailability (absorbed more rapidly and to a higher degree) which can be illustrated by an increased equilibrium solubility, rate of dissolution and solubility in comparison to a xanthine oxidase inhibitor alone.

In one aspect, the rate of dissolution (i.e. mass of substance dissolved in a defined time period) of a xanthine oxidase inhibitor may be increased up to several fold in a composition of the invention when compared to the pure active substances. The solubility (i.e. mass of substance having dissolved clearly in a mass or certain volume of solvent) of a xanthine oxidase inhibitor contained in a composition of the invention may be increased giving rise to supersaturated solutions. An increase in terminal solubility may result which is maintained for at least several hours then decreasing to the solution's degree of saturation. In an embodiment, provided is a composition with a resorption rate increased by a factor of 1.5, 2, 3, 4, 5, 10, 15, 20, and 50 when compared to the pure active substances.

In another embodiment, the invention provides a composition, comprising a xanthine oxidase inhibitor that induces a decreased intracellular uric acid concentration. The decrease in circulating uric acid levels, or intracellular uric acid concentrations may represent at least about a 1%, 5%, 10%, 15%, 20%, 30%, 40%, 50%, 60%, 70% or, 1, 2, 3, 10, 30 or 100 fold decrease in circulating uric acid levels, or intracellular uric acid concentration in an in vitro uric acid assay or in vivo measurement of serum uric acid or intracellular or tissue uric acid.

In particular embodiments of the invention, the organic base includes but is not limited to arginine, choline, L-lysine, D-lysine, glucamine and its N-mono- or N,N-disubstituted derivatives including but not limited to N-methylglucamine, N,N-dimethylglucamine, N-ethylglucamine, N-methyl,N-ethylglucamine, N,N-diethylglucamine, N-β-hydroxyethylglucamine, N-methyl,N-β-hydroxyethylglucamine, and N,N-di-β-hydroxyethylglucamine, benethamine, banzathine, betaine, deanol, diethylamine, 2-(diethylamino)-ethanol, hydrabamine, 4-(2-hydroxyethyl)-morpholine, 1-(2-hydroxyethyl)-pyrrolidine, tromethamine, diethanolamin (2,2″-iminobis(ethanol), ethanolamine(2-aminoethanol), 1H-imidazole, piperazine, triethanolamine (2,2′,2″-nitrilotris (ethanol), N-methylmorpholine, N-ethylmorpholine, pyridine, dialkylanilines, diisopropylcyclohexylamine, tertiary amines (e.g. triethylamine, trimethylamine), diisopropylethylamine, dicyclohexylamine, N-methyl-D-glutamine, 4-pyrrolidinopyridine, dimethylaminopyridine (DMAP), piperidine, isopropylamine, or caffeine. In other embodiments, the organic base is a biguanide or biguanidine such as metformin. In further embodiments, the organic base is a gliflozin.

In another embodiment, the organic base is a basic amino acid, in particular lysine and arginine and the xanthine oxidase inhibitor is allopurinol or oxypurinol. In an embodiment, a liquid composition is provided comprising allopurinol or oxypurinol and L-arginine.

In a further embodiment, the composition of xanthine oxidase inhibitor and basic amino acid, may be administered as a powder to an animal or human subject, the enhanced solubility of the composition upon contact with water or other aqueous solution, or food material in the gastrointestinal track being sufficient to generate the liquid dosing form.

In a further embodiment, the invention provides compositions, especially liquid compositions comprising xanthine oxidase inhibitors and choline. Choline is a physiological compound which has been used in therapy and it does not suffer from disadvantages such as systemic or local toxicities.

An XOI and organic base can be in a ratio selected to augment the solubility of the XOI, augment the activity of the XOI, or provide a beneficial effect. The ratio of organic base to xanthine oxidase inhibitor can range from about 0.01 to 20.0 molar equivalent organic base to 1.0 molar equivalent of XOI. In an embodiment the ratio of organic base to XOI is 1.0:0.5 mole, in particular 1.0:1.0 mole, more particularly 1.0:3.0 mole. In a specific embodiment, the composition comprises oxypurinol as the XOI and metformin as the organic base, wherein the ratio of oxypurinol to metformin is 1:01 to 1:10 moles.

A composition of the invention may also comprise a pharmaceutically acceptable carrier, excipient, vehicle, or diluent. A XOI and organic base can be mixed into a selected pharmaceutically acceptable carrier, excipient, vehicle, or diluent, and optionally other active ingredients including therapeutic agents are added.

The compositions of the present invention typically comprise suitable pharmaceutical carriers, excipients, vehicles, or diluents selected based on the intended form of administration, and consistent with conventional pharmaceutical practices. Suitable pharmaceutical carriers, excipients, vehicles, or diluents are described in the standard text, Remington's Pharmaceutical Sciences (Mack Publishing Company, Easton, Pa., USA 1985). By way of example, for oral administration in a liquid form, the drug components (i.e. XOI and organic base) may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, targeting agents, coloring agents, and other agents known to one skilled in the art, may also be combined in the compositions or components thereof.

In an embodiment, a composition of the invention is formulated so that it remains active at physiologic pH. The composition may be formulated in the pH range 4 to 10, in particular 5 to 9.

In an aspect, the invention relates to an aqueous composition comprising a XOI of the invention and a solvent system which effects solubilization of the inhibitor. The solvent system effects solubilization of the inhibitor to provide an aqueous solution with beneficial properties for incorporation into an oral liquid formulation. The solvent system comprises an organic base, in particular arginine or lysine, more specifically arginine.

In an aspect, a liquid compositions may be prepared using an XOI, in particular allopurinol or oxypurinol and arginine, choline, glucamine (n-methylglucamine), or glucamine salts.

In an aspect, the invention provides a sterile, pyrogen-free, ready-to-use solution of a XOI, especially allopurinol or oxypurinol, which consists essentially of the XOI and organic base dissolved in a physiologically acceptable solvent therefore. In an embodiment, the solution has not been reconstituted from a lyophilizate.

The invention also provides an orally applicable composition comprising a XOI and organic base dissolved in a physiologically acceptable solvent therefor.

In a particular liquid composition of the invention comprising oxypurinol and arginine the concentration of oxypurinol is about 0.1-100 mg/ml, 0.5-50 mg/ml, 1-25 mg/ml, and 1-10 mg/ml, more preferably 10 mg/ml. The solution may be administered in a total volume of about 5 to 100 ml, preferably 30 ml, twice a day, to achieve the desired dose of about 200 to 1000 mg/day, preferably 600 mg/day. Alternatively, if a once-a-day dosing regimen is desired, the concentration of oxypurinol in the formulation may be about 6.0 to 60 mg/ml, preferably 20 mg/ml, administered in about 10 to 100 ml, preferably 30 ml of solution once a day.

Preferred liquid formulations of the invention comprise:
(1) allopurinol, oxypurinol, febuxostat or tautomers thereof in a concentration range of about 0.1 to about 100 mg/ml (w/v), more preferably about 0.5 to about 50 mg/ml (w/v), even more preferably about 1 to about 25 mg/ml (w/v), still more preferably about 1 to 10 mg/ml (w/v), and most preferably about 10 mg/ml; (2) a basic amino acid having an appropriate concentration range to provide the suitable mole ratios of basic amino acid to XOIs or metformin; and (3) a pharmaceutically acceptable diluent, preferably water, to bring the composition to a designated volume.

The invention contemplates a lyophilized formulation as described herein. The lyophilization composition of the present invention can provide a product with increased stability, solubility or bioavailability. Lyophilized formulations of XOIs comprise: a XOI, an organic base, and a pharmaceutically acceptable carrier, excipient, or diluent. Storage conditions for the lyophilized formulation are typically at about 2° C. to about 25° C. XOIs with an organic base (e.g. arginine) can be lyophilized at a concentration of about 0.02 mg/ml to about 10 mg/ml of compound in the initial solution. A lyophilization solution preferably comprises (in addition to the XOIs, an organic base, and a lyophilization buffer. The preferred pH range for the lyophilization buffer is from about 5.5 to about 12.0. A lyophilization buffer may contain sodium citrate, EDTA, and/or sucrose. A lyophilized xanthine oxidase formulation can be reconstituted in sterile water so as to maintain isotonic conditions of about 290 mOsm. The XOIs with organic base can be reconstituted in sterile water, optionally containing a stabilizing amount of antioxidants.

### EXAMPLES

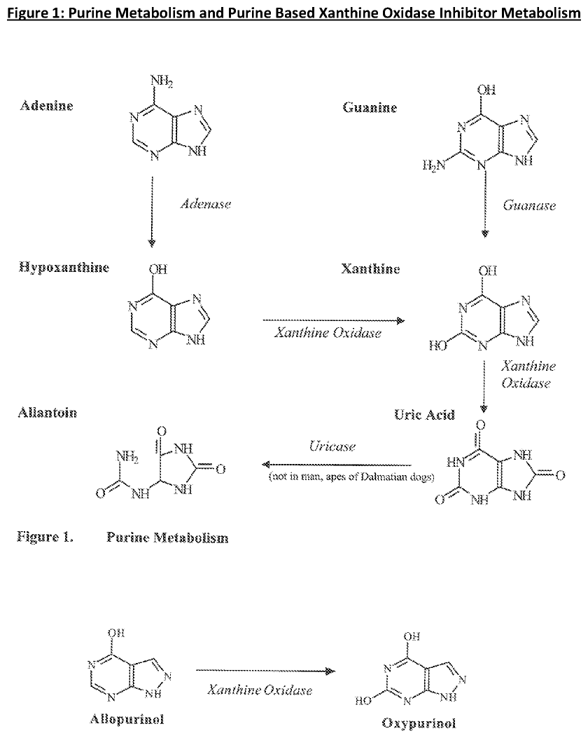

FIG. 1: provides a schematic depicting purine metabolism and purine based xanthine xxidase inhibitor metabolism. Xanthine oxidase and xanthine dehydrogenase participate in enzymatic conversion of hypoxanthine to xanthine, then to uric acid. In many other life forms, except in man, apes and dalmation dogs, uricase enzyme is active and further converts uric acid to allantoin. Aldehyde oxidase and xanthine oxidase enzymes rapidly convert allopurinol into oxypurinol.

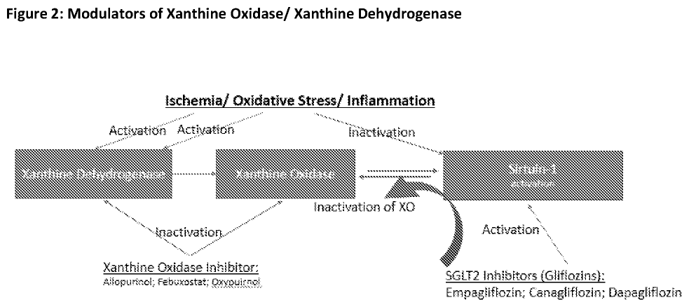

FIG. 2: provides a diagram depicting modulators of Xanthine Oxidase/Xanthine Dehydrogenase. Ischemia, oxidative stress and inflammation are associated with increased xanthine oxidase activity and decrease sirtuin-1 activity. In disease, xanthine oxidase activity and activation results in increased uric acid and oxygen radical production. Intracellular and extracellular inhibition of xanthine oxidase activity may decrease injury to tissue. SGLT2 inhibitors activate sirtuin-1 activity which in turn decreases activity of xanthine oxidase. SGLT2 inhibition may indirectly protect cystic tissue that overexpresses xanthine oxidase by suppressing activity of xanthine oxidase, decreasing uric acid and oxygen free radicals in tissue. Both xanthine oxidase inhibition and SGLT2 inhibition may exert their protective effect in cystic tissue. SGLT2 inhibitors increase Sirtuin1 activity which inactivates Xanthine Oxidase activity thereby acting as a uric acid lowering agent. SGLT2 inhibitors have also been reported to lower uric acid by decreasing kidney and intestinal uric acid reabsorption.

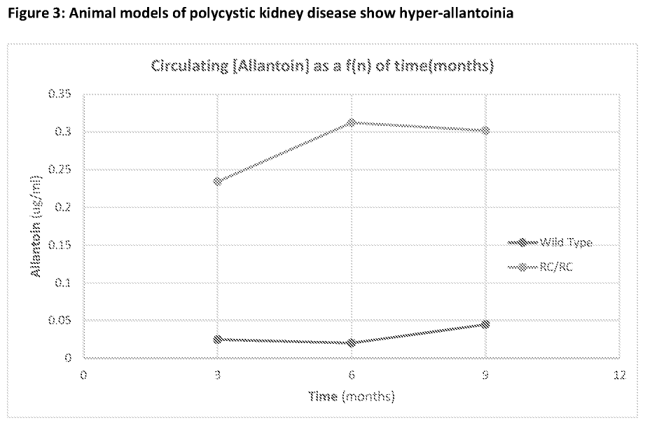

FIG. 3: provides a graph showing that animal models of polycystic kidney disease exhibit high serum concentrations of allantoin. Hyperallantoinia was previously reported in a Han/SPRD rat model of polycystic kidney disease (Edelstein et al). Hyper-allantoinia occurs early in mouse models of polycystic kidney disease and persists from as early as 3 months through 9 months in the RC/RC mouse. Because allantoin is the product of uric acid breakdown by the uricase enzyme, this finding supports the concept that high chronic exposure to uric acid is ongoing in polycystic kidney disease and may contribute to the health consequences of and progression of declining kidney structure and function.

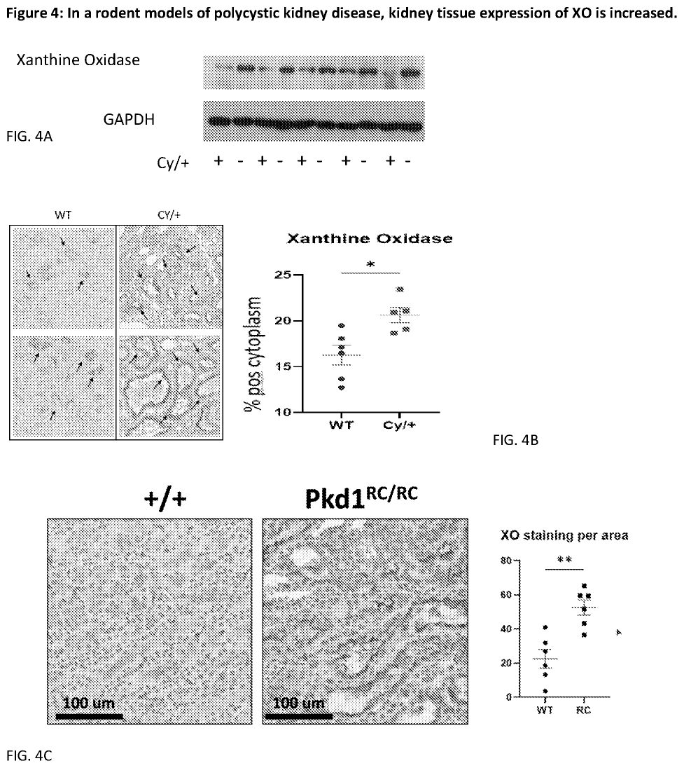

FIG. 4: provides data showing that kidney tissue expression of XO is increased in a rodent model of polycystic kidney disease. Identification and quantification of xanthine oxidase in kidney tissue, in wild type (WT) and progressing PKD rat CY/+, and in RC/RC mice was conducted in late stage polycystic kidney disease. Statistically significant (*; **) increase in xanthine oxidase protein concentration was observed using western blot. FIG. 4A provides a western blot showing increased expression of xanthine oxidase. FIG. 4B Provides Data from a rat model of polycystic kidney disease Han/SPDR (CY/+) or wild type rat kidney. Light microscopy of kidney tissue sections, stained to show the presence of the xanthine oxidase enzyme, and reads as relative densitometric Units (RD). Han/SPDR show an increased proportion of cytoplasm stained compared to wildtype (p<0.05). FIG. 4C Provides evidence of a similar observation in the mouse polycystic kidney disease model (Pkd1 RC/RC mouse) (P<0.05).

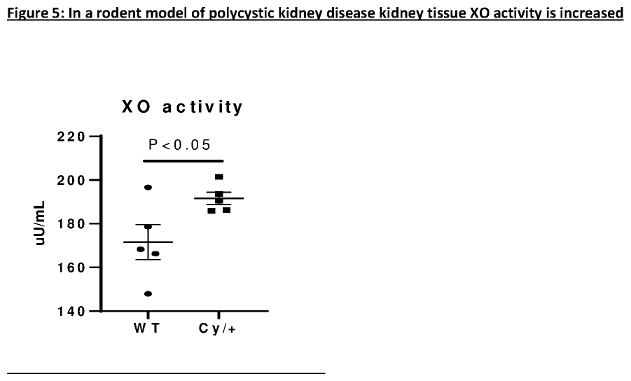

FIG. 5: is a graph showing that XO activity is increased in a rodent model of polycystic kidney disease kidney tissue compared to wild-type. Quantification of xanthine oxidase activity in kidney tissue, in wild type (WT) and progressing PKD rat CY/+, was conducted in late stage polycystic kidney disease. Statistically significant increase in xanthine oxidase activity was observed in the Han/SPDR rat.

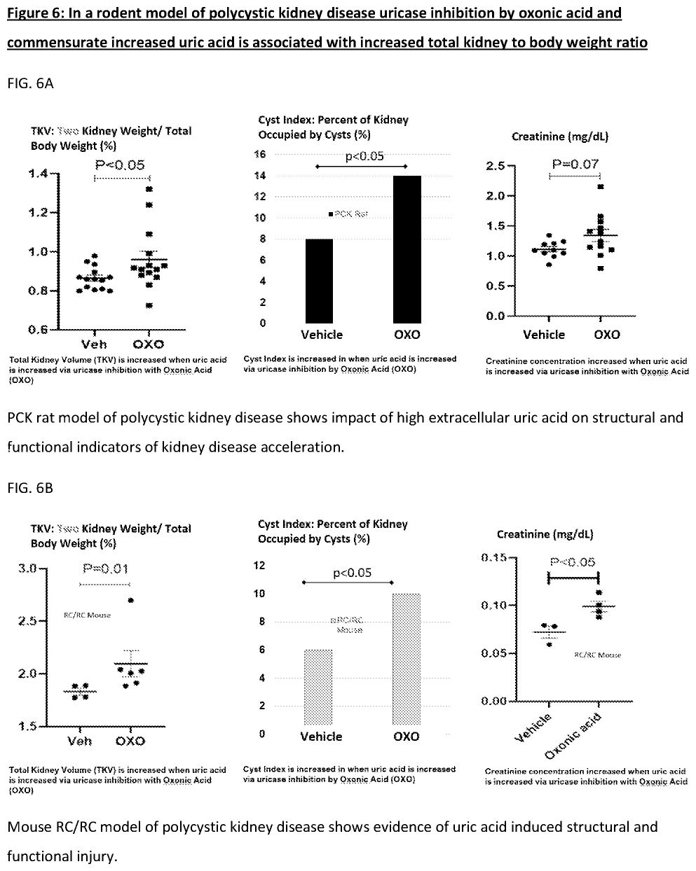

FIG. 6: provides data showing that uricase inhibition by oxonic acid (OXO) and commensurate increased uric acid is associated with increased total kidney to body weight ratio (2K/BWT) in a rat model of polycystic kidney disease (PCK) and in a mouse model of polycystic kidney disease (Pkd1RC/RC). Rodent models of polycystic kidney disease were treated with a uricase enzyme inhibitor—oxonic acid (OXO)—to decrease the metabolism of uric acid to allantoin, thereby chronically increasing circulating serum uric acid concentration. Following 8 weeks of treatment with oxonic acid, total kidney volume is increased significantly compared to untreated animals. After 70 days exposure to oxonic acid both models showed similar results, increased of serum uric acid resulted in an increase in 2K/BWT, increased cyst sized and increased serum creatinine (P<0.05; P<0.05; for rat and mouse creatinine P<0.07 & P<0.05). Increased serum uric acid is associated with structural and functional changes in kidney function. Increased creatinine is associated with decreased filtering capacity of kidneys. disease. This result suggests that uric acid modulates cyst genesis and cyst growth rate, thereby modulating kidney structure and disease progression. FIG. 6A shows the data from the PCK rat model and FIG. 6B shows the data from the mouse RC/RC model.

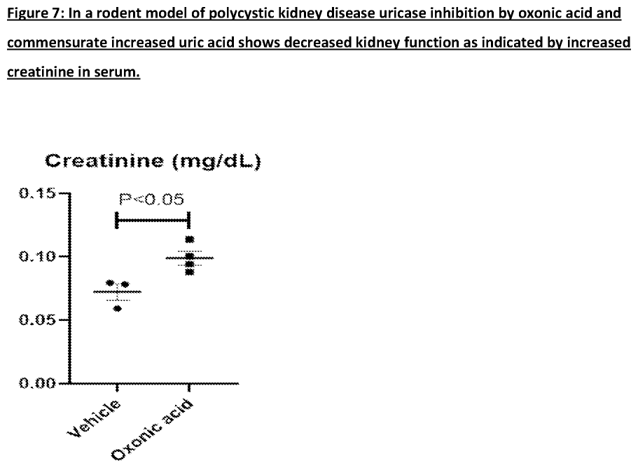

FIG. 7: provides data showing that in a rodent model of polycystic kidney disease uricase inhibition by oxonic acid and commensurate increased uric acid shows decreased kidney function as indicated by increased creatinine in serum.

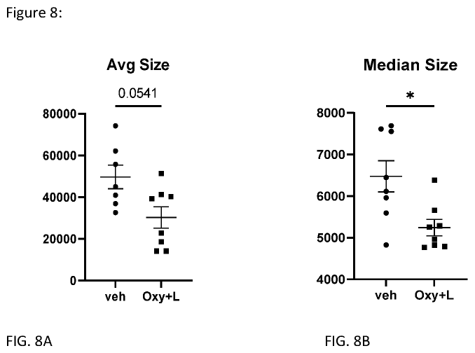

FIG. 8: In a rodent model of polycystic kidney disease-mouse RC/RC model—xanthine oxidase inhibition attenuates kidney disease progression as indicated by average total kidney volume (FIG. 8A) and median size of cysts in the kidney (FIG. 8B). This suggests that that inhibition of uric acid production by xanthine oxidase can attenuate the renal size in polycystic kidney disease, compared to placebo (veh).

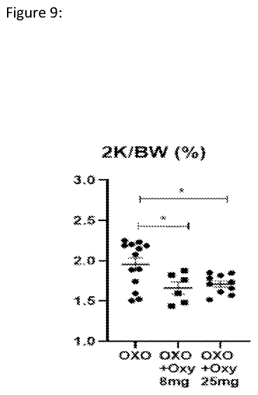

FIG. 9: is a graph showing that in a rodent model of polycystic kidney disease—mouse RC/RC-oxonic acid (OXO) exposure increased kidney size. When OXO and Xanthine oxidase inhibition (OXY+L) are administered simultaneously, OXY+L attenuated the oxonic acid effect and normalized/decreased uric acid induced kidney expansion at a moderate (8 mg/kg/day), or at a higher oral dose (24 mg/kg/day). This data shows that detrimental effects of high uric acid can be alleviated by reducing uric acid levels using UALAs.

What is claimed is:

1. A method of detecting and treating kidney disease progression in a subject, the method comprising
obtaining a sample from a subject; and
detecting a level of xanthine oxidase expression or activity in a sample, and if the level of xanthine oxidase expression or activity is at or above a predetermined level, or is elevated relative to a control, determining the subject is a subject in need; and
administering a therapeutically effective amount of xanthine oxidase inhibitor to the subject in need.

2. The method of claim 1, wherein the kidney disease progression is polycystic kidney disease progression.

3. The method of claim 2, wherein the polycystic kidney disease progression is autosomal dominant or autosomal recessive polycystic kidney disease.

4. The method of any of claims 1-3, wherein the sample comprises a body fluid, wherein the body fluid is optionally blood, plasma, serum, tears, perspiration, urine, fecal matter, or tissue.

5. The method of any of claims 1-4, further comprising detecting uric acid concentration in the sample or another sample from the subject, wherein uric acid concentration at or above a predetermined level, or elevated relative to a control further indicates kidney disease progression.

6. The method of any of claims 1-5, wherein the sample is blood, urine or both and the detecting comprises detecting antigens associated with XO or XDH or both, or amino acid chain fragments of XO or XDH. 7, The method of any of claims 1-6, wherein the xanthine oxidase inhibitor comprises oxypurinol. 8 The method of claim 7, wherein the composition further comprises an organic base, wherein the organic base is optionally one or more of L-arginine, D-arginine, choline, L-lysine, D-lysine, caffeine, biguanide, biguanidine or gliflozin.

9. The method of claim 7, further comprising co-administering a therapeutically effective amount of an organic base.

10. A method comprising detecting changes to xanthine oxidase (XO) concentration or activity or xanthine dehydrogenate (XDH) concentration or activity, or the XO/XDH ratio of concentration or activity, the method comprising
obtaining a sample from a subject exhibiting one or more symptoms of polycystic kidney disease, wherein the sample comprises a blood sample or urine sample, or extracellular vesicle sample from the blood sample or the urine sample; and
detecting XO concentration or activity and XDH concentration or activity in the sample; and
administering a UALA and, optionally co-administering an organic base, if the sample comprises XO concentration or activity, XDH concentration or activity, and/or an XO/XDH ratio of concentration or activity that is a deviation from that of a healthy subject.

11. The method of claim 10, wherein a deviation from that of a healthy subject is
a XO concentration above 1 mg/L;
XO enzyme activity above 105 (U/L); and/or
a XO/XDH that is at least 0.1-10% disparate with that of a healthy subject.

12. A method of reducing aberrant purine metabolism associated with kidney disease in a subject in need, the method comprising administering a therapeutically effective amount of one or more uric acid lowering agents, wherein the method treats a symptom of cystic disease, and wherein the one or more uric acid lowering agents are optionally selected from the group consisting of xanthine oxidase inhibitor and sirtuin-1 activator.

13. The method of claim 12, wherein the subject in need has one or more cysts present in a kidney or liver.

14. The method of claim 12, wherein administering comprises co-administering a oxypurinol and a gliflozin.

15. A method of reducing markers of kidney disease progression in a subject, the method comprising obtaining a sample from a subject; and
detecting a marker for kidney disease, the marker comprising tissue oxygen radicals, uric acid, cytokines, inflammatory cells, fibrosis, mitochondriosis, or Sirtuin-1 in a sample, and
if the level of marker is above baseline, administering a therapeutically effective amount of UALA and, optionally, co-administering an organic base, to the subject in need.

16. The method of claim 15, wherein the kidney disease progression is polycystic kidney disease progression.

17. The method of claim 15 or 16, wherein the UALA is an interfering molecule that targets xanthine oxidase or xanthine dehydrogenase expression, or is an eRNA that increases expression of sirtuin-1.

18. The method of claim 17, wherein administering comprises co-administering a xanthine oxidase inhibitor and an interfering molecule that targets xanthine oxidase or xanthine dehydrogenase expression.

19. A method comprising detecting presence of a marker, or a ratio of markers in a biological sample from a subject; and if the marker is elevated relative to a baseline or control, or if a ratio of markers is disproportionate to a baseline or control, administering a therapeutically effective amount of at least one uric acid lowering agent to the subject.

20. The method of claim 19, wherein the marker is one or more of xanthine oxidase, xanthine dehydrogenase, a sirtuin or Sirtuin-1, Hypoxia Inducing Factor-1 (HIF-1), Erythropoietin, PCNA, Wnt/B-Catenin, IL-5, IL-6, STAT1, STAT2, mTOR, TNFa, MIF, NLRP3 Inflammasome, or constituents of blood or urine borne cell membranes, micro-vesicles, apoptotic bodies, exosomes, or free enzymes or specific portions/fragments of enzymes.

21. The method of claim 20, wherein the ratio comprises a xanthine oxidase/xanthine dehydrogenase ratio.

22. The method of claim 20, wherein the uric acid lowering agent is a xanthine oxidase inhibitor, or an interfering molecule targeting expression of XO or XDH in cells of the subject.

23. The method of any of claims 20-22, further comprising administering a conjunctive agent.

24. The method of claim 23, wherein the conjunctive agent is a vasopressin receptor antagonist.

25. A method comprising administering a therapeutically effective amount of a uric acid lowering agent to a subject exhibiting elevated xanthine oxidase activity or concentration relative to that of a healthy subject, or a XO/XDH concentration or activity ratio that deviates from that of a healthy subject, in a urine sample of the subject.

26. The method of claim 25, further comprising administering a conjunctive agent.

27. A method of monitoring effectiveness of a treatment of polycystic kidney disease comprising
administering an amount of a uric acid lowering agent to a subject exhibiting elevated xanthine oxidase activity or concentration or elevated xanthine oxidase/xanthine dehydrogenase activity or concentration ratio in urine in a subject relative to baseline; and
detecting xanthine oxidase concentration or activity or xanthine oxidase/xanthine dehydrogenase concentration or activity ratio in urine in a subject, wherein a decrease in xanthine oxidase activity or concentration or a migration of the xanthine oxidase/xanthine dehydrogenase concentration or activity ratio toward baseline indicates effectiveness of treatment.

28. A composition comprising an amount of a oxypurinol and an amount of metformin, and/or an SGLT2 Inhibitor.

29. The composition of claim 28, wherein the SGLT2 inhibitor is a gliflozin.

30. The composition of claim 28, comprising oxypurinol, metformin and gliflozin.

31. A method of treating polycystic kidney disease that comprises administering a therapeutically effective amount of the composition of any of claims 28-30.

32. A method of treating polycystic kidney disease that comprises co-administering therapeutically effective amounts of xanthine oxide inhibitor and metformin.

33. A composition comprising a therapeutically effective amount of a uric acid lowering agent and a therapeutically effective amount of tolvaptan and/or lixivaptan.

34. The composition of claim 33, wherein the uric acid lowering agent is a xanthine oxidase inhibitor.

35. The method of claim 34, wherein the xanthine oxidase inhibitor is oxypurinol.

36. A method of treating polycystic kidney disease that comprises administering a therapeutically effective amount of the composition of any of claims 33-35.

37. A method of treating polycystic kidney disease that comprises co-administering therapeutically effective amounts of xanthine oxide inhibitor and tolvaptan and/or lixivaptan.

38. A method of treating cystic disease in a subject in need, the method comprising co-administering a therapeutically effective amount of a uric acid lowering agent and gliflozin, wherein gliflozin is co-administered in an amount to increase the aqueous solubility and bioavailability of the uric acid lowering agent.

39. A method of treating a cystic disease, the method comprising co-administering a therapeutically effective amount of a xanthine oxidase inhibitor and a Sirtuin-1 activator.

40. The method of claim 39, wherein the sirtuin-1 activator is a SGLT2 inhibitor.

41. The method of claim 39 or 40, wherein the method ameliorates genesis or growth of cyst.

42. The method of any of claims 39-41, wherein the cystic disease is polycystic kidney disease.

43. A method of treating cystic disease in a subject in need, the method comprising co-administering a therapeutically effective amount of at least one xanthine oxidase inhibitor and an agent that directly or indirectly decreases expression of xanthine oxidase.

44. The method of claim 43, wherein the agent that directly decreases expression of xanthine oxidase comprises an interfering molecule targeting xanthine oxidase expression.

45. The method of claim 43, wherein the agent that indirectly decreases expression of xanthine oxidase comprises a sirtuin-1 activator.

46. The method of claim 45, wherein the sirtuin-1 activator comprises a gliflozin or an eRNA that induces expression of sirtuin-1, or a combination thereof.

47. A composition comprising a therapeutically effective amount of a xanthine oxidase inhibitor and an agent that directly or indirectly decreases expression of xanthine oxidase.

48. The composition of claim 47, wherein the agent that directly decreases expression of xanthine oxidase comprises an interfering molecule targeting xanthine oxidase expression.

49. The composition of claim 47, wherein the agent that indirectly decreases expression of xanthine oxidase comprises a sirtuin-1 activator.

50. The composition of claim 49, wherein the sirtuin-1 activator comprises a gliflozin or an eRNA that induces expression of sirtuin-1, or a combination thereof.
